(19) United States
(12) Patent Application Publication
(10) Pub. No.: US 2022/0403328 A1
(43) Pub. Date: Dec. 22, 2022

(54) METHOD AND KIT FOR EXPANDING CIRCULATING TUMOR CELLS EX VIVO, COMPOSITE MATERIAL FILM AND PREPARATION METHOD THEREOF, DRUG TESTING METHOD, AND CRYOPRESERVATION SOLUTION

(71) Applicant: Cancer Free Biotech Ltd., Taipei City (TW)

(72) Inventors: Po-Han Chen, Taipei City (TW); Wei-Hsin Hsu, Taipei City (TW); Shih-Pei Wu, Taipei City (TW)

(21) Appl. No.: 17/772,223

(22) PCT Filed: Nov. 5, 2020
(86) PCT No.: PCT/CN2020/126634

§ 371 Date: Apr. 27, 2022

Related U.S. Application Data
(60) Provisional application No. 62/931,236, filed on Nov. 6, 2019.

(51) Int. Cl. C12N 5/00; A01N 1/02; C12N 5/09

(52) U.S. Cl. CPC C12N 5/0068; A01N 1/0221; C12N 5/0693; C12N 2533/20; C12N 2533/40

(57) ABSTRACT

A composite material film for expanding circulating tumor cells ex vivo and a preparation method thereof, a kit and a method for expanding circulating tumor cells ex vivo, a method for detecting an effect of a drug, and a cryopreservation solution are provided. The preparation method includes: mixing one or more kinds of particles and a solvent to form a mixed liquid, in which the particles are selected from the group consisting of metal particles, metal oxide particles, silicon oxide particles and combinations thereof; placing the mixed liquid on a substrate to form a particle layer; adding a medium material to the particle layer, in which the medium material is selected from the group consisting of styrene and its derivatives, polyester monomers, silicon oxide compounds and combinations thereof; and polymerizing the medium material to form a medium layer to fix the particle layer on the substrate.

### FIELD OF THE INVENTION

The present disclosure relates to the technical field of expanding circulating tumor cells, in particular to a composite material film for expanding circulating tumor cells ex vivo, a method for preparing a composite material film for expanding circulating tumor cells ex vivo, a method for expanding circulating tumor cells ex vivo, a kit for expanding circulating tumor cells ex vivo, a method for detecting an effect of a drug, and a cryopreservation solution.

### BACKGROUND OF THE INVENTION

When cancer cells are detached from in situ tumor cells into the circulatory system (e.g., blood), these cancer cells in the blood are called as circulating tumor cells (CTCs). CTC counting is an emerging cancer biomarker, and many studies have confirmed that this method can predict a prognosis of cancer, and a number of cells is monitored as an indicator for a patient's response to chemotherapy and targeted therapy. At present, most of relevant clinical applications use the CTC counting to judge progression of a disease. However, although a few research papers have theoretically confirmed that CTCs can reflect the patient's treatment response to drug in real time and directly, the number of CTCs obtained by this method is very limited and cannot be widely used. The main reason is that due to lack of suitable technology to expand CTCs, a small number of CTCs cannot be used for accurate genetic testing and drug sensitivity testing with sufficient samples. Moreover, a success rate of ex vivo culture of CTCs is quite low (less than 20%) and takes more than six months, which limits its clinical application. Breaking through the bottleneck in the number of CTCs has been regarded as the most urgent research project for tumor metastasis research and clinical application.

### SUMMARY OF THE INVENTION

Therefore, embodiments of the present disclosure discloses a composite material film for expanding circulating tumor cells ex vivo, a method for preparing a composite material film for expanding circulating tumor cells ex vivo, a method for expanding circulating tumor cells ex vivo, a kit for expanding circulating tumor cells ex vivo, a method for detecting an effect of a drug, and a cryopreservation solution, which can effectively expand the circulating tumor cells.

Specifically, in a first aspect, the embodiments of the present disclosure discloses a method for preparing a composite material film for expanding circulating tumor cells ex vivo, which includes: mixing one or more kinds of particles and a solvent to form a mixed liquid, in which the particles are selected from the group consisting of metal particles, metal oxide particles, silicon oxide particles and combinations thereof; placing the mixed liquid on a substrate to form a particle layer; adding a medium material to the particle layer, in which the medium material is selected from the group consisting of styrene and its derivatives, polyester monomers, silicon oxide compounds and combinations thereof; and polymerizing the medium material to form a medium layer to fix the particle layer on the substrate.

In one embodiment of the present disclosure, the metal particles are selected from the group consisting of gold particles, silver particles, titanium particles and combinations thereof, and the metal oxide particles are titanium dioxide particles, and the silicon oxide particles are selected from the group consisting of silicon dioxide particles, silica gel particles, polydimethylsiloxane particles and combinations thereof.

In one embodiment of the present disclosure, a particle size of the one or more kinds of particles is in a range of between 10 nanometers and 10 micrometers.

In one embodiment of the present disclosure, the method further includes performing hydrophilization pretreatment on the substrate before placing the mixed liquid on the substrate, in which the hydrophilization pretreatment includes surface plasma treatment, hydrophilic polymer coating, acid or alkaline rinse or a combination thereof.

In one embodiment of the present disclosure, the method further includes performing standing treatment after placing the mixed liquid on the substrate to make the one or more kinds of particles of the mixed liquid self-assemble and arrange to form the particle layer.

In one embodiment of the present disclosure, the method further includes performing drying treatment after performing the standing treatment, and the drying treatment includes dehumidification drying, reduced pressure drying, heating drying or a combination thereof.

In one embodiment of the present disclosure, the styrene derivatives include carboxylated styrene, styrene sulfonic acid or a combination thereof, and the polyester monomers include methylmethacrylate.

In one embodiment of the present disclosure, the silicon oxide compounds are selected from the group consisting of polydimethylsiloxane, tetraethoxysilane and combinations thereof.

In a second aspect, the embodiments of the present disclosure discloses a composite material film for expanding circulating tumor cells ex vivo, which includes: a particle layer including one or more kinds of particles substantially regularly arranged, in which the one or more kinds of particles are selected from the group consisting of metal particles, metal oxide particles, silicon oxide particles and combinations thereof; and a medium layer disposed between the one or more kinds of particles of the particle layer, in which the medium layer is selected from the group consisting of polystyrene and its derivatives, polyester, silicon dioxide, silica gel, silicone, silicone rubber and combinations thereof, in which surfaces of some of the one or more kinds of particles are partially exposed and not covered by the medium layer.

In a third aspect, the embodiments of the present disclosure discloses a kit for expanding circulating tumor cells ex vivo, which includes: a culture vessel, including: a substrate; and the composite material film prepared by the above-mentioned method, attached to the substrate; and a culture medium, including a stem cell culture medium.

In a fourth aspect, the embodiments of the present disclosure discloses a method for expanding circulating tumor cells ex vivo, which includes: mixing a plurality of circulating tumor cells with a culture medium to form a cell fluid; and contacting the cell fluid to the composite material film prepared by the above-mentioned method to allow the circulating tumor cells to attach the one or more kinds of particles and expand.

In a fifth aspect, the embodiments of the present disclosure discloses a cryopreservation solution for cryopreserving expanded circulating tumor cells, which includes: a frozen reagent; and a culture medium, including a basic fibroblast growth factor (bFGF) and an epidermal growth factor (EGF).

The above technical solutions have following advantages or beneficial effects: the composite material film provided by the present disclosure is suitable as a base for circulating tumor cells to attach and expand and can be applied to effectively increase a number of the circulating tumor cells.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In order to illustrate technical solutions of embodiments of the present disclosure more clearly, the following briefly introduces accompanying drawings used in description of the embodiments. Obviously, the drawings in the following description are only some embodiments of the present disclosure. For a person skilled in the art, other drawings can also be obtained from these drawings without any creative effort.

FIG. 1 is a flow chart of a method for preparing a composite material film for expanding circulating tumor cells ex vivo according to some embodiments of the present disclosure.

FIG. 2 is a schematic diagram of steps of a method for preparing a composite material film for expanding circulating tumor cells ex vivo according to some embodiments of the present disclosure.

FIG. 3 is a flow chart of a method for expanding circulating tumor cells ex vivo according to some embodiments of the present disclosure.

FIG. 4 is a schematic diagram of steps of a method for expanding circulating tumor cells ex vivo according to some embodiments of the present disclosure.

FIG. 5 is an image of a composite material film of Experimental Example 1 of the present disclosure.

FIG. 6A is a SEM image of a material film of Comparative Example 1 of the present disclosure.

FIG. 6B is a SEM image of a composite material film of Experimental Example 1 of the present disclosure.

FIG. 7A is an optical microscope image of cell morphology of breast cancer cells after being cultured on the material film of Comparative Example 1.

FIG. 7B is an optical microscope image of a cell fluid collected in a clean culture plate after the breast cancer cells cultured on the material film of Comparative Example 1 is rinsed.

FIG. 8A is an optical microscope image of cell morphology of breast cancer cells after being cultured on the composite material film of Experimental Example 1.

FIG. 8B is an optical microscope image of a cell fluid collected in a clean culture plate after the breast cancer cells cultured on the composite material film of Experimental Example 1 is rinsed.

FIG. 9 shows characterization and identification of staining results of circulating tumor cells of a lung cancer patient after has been cultured on the composite material film of Experimental Example 1 of the present disclosure for the fourth week.

FIG. 10 shows another characterization and identification of staining results of the circulating tumor cells of the lung cancer patient after has been cultured on the composite material film of Experimental Example 1 of the present disclosure for the fourth week.

FIG. 11 shows characterization and identification of staining results of circulating tumor cells of a gastric cancer patient after has been cultured on the composite material film of Experimental Example 1 of the present disclosure for the fourth week.

FIG. 12 is a graph showing comparison of cell viability counts after circulating tumor cells of a lung cancer patient and circulating tumor cells of an ovarian cancer patient have been cultured on the material film of Comparative Example 1 and the composite material film of Experimental Example 1 for four weeks.

### DETAILED DESCRIPTION

Technical solutions in embodiments of the present disclosure will be clearly and completely described below with reference to accompanying drawings in the embodiments of the present disclosure. Obviously, the described embodiments are only some, but not all, of the embodiments of the present disclosure. Based on the embodiments of the present disclosure, all other embodiments obtained by a person skilled in the art without creative efforts shall fall within a protection scope of the present disclosure.

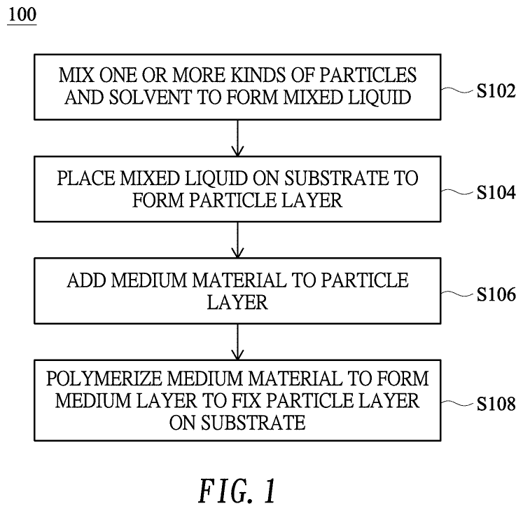

The present disclosure provides a method for preparing a composite material film for expanding circulating tumor cells ex vivo. FIG. 1 is a flow chart of a method 100 for preparing a composite material film for expanding circulating tumor cells ex vivo according to some embodiments of the present disclosure. As shown in FIG. 1, the method 100 for preparing the composite material film includes following steps: mixing one or more kinds of particles and a solvent to form a mixed liquid (Step S102), placing the mixed liquid on a substrate to form a particle layer (Step S104), adding a medium material to the particle layer (Step S106) and polymerizing the medium material to form a medium layer to fix the particle layer on the substrate (Step S108).

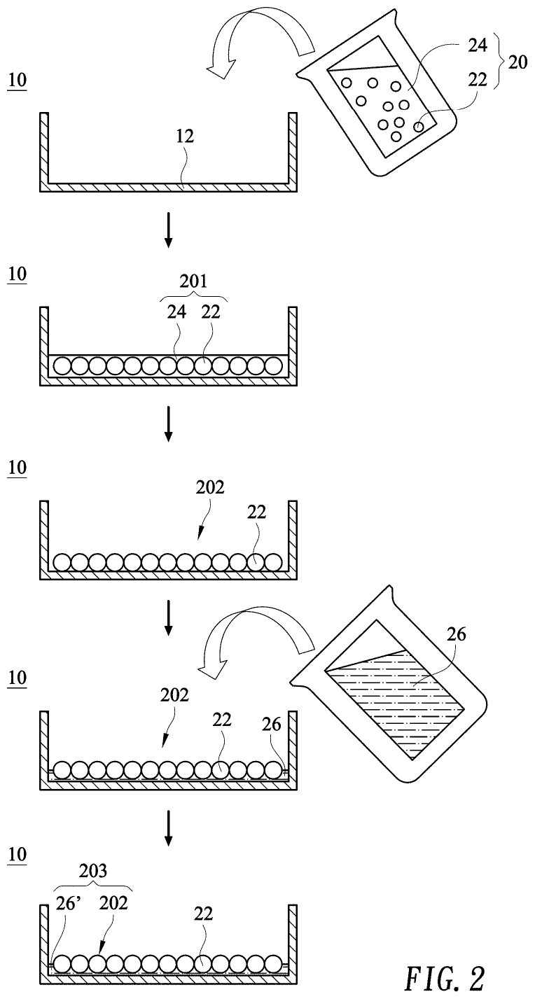

FIG. 2 is a schematic diagram of steps of a method for preparing a composite material film for expanding circulating tumor cells ex vivo according to some embodiments of the present disclosure. For the following embodiments, please refer to FIG. 1 and FIG. 2 simultaneously.

First, one or more kinds of particles 22 and a solvent 24 are mixed to form a mixed liquid 20 (Step S102). The aforementioned one or more kinds of particles are selected from the group consisting of metal particles, metal oxide particles, silicon oxide particles, and combinations thereof. In some embodiments, the metal particles include gold particles (Au particles), silver particles (Ag particles), titanium particles (Ti particles), other suitable metal particles or a combination thereof. The metal oxide particles include titanium dioxide particles, other suitable metal oxide particles or a combination thereof. The silicon oxide particles include silicon dioxide particles, silica gel particles, polydimethylsiloxane particles, other suitable silicon oxide particles or a combination thereof. In some embodiments, a particle size of each of the one or more particles is in a range of between 10 nanometers and 10 micrometers, or between 400 nanometers and 10 micrometers, or between 500 nanometers and 10 micrometers, or between 1 micrometer and 10 micrometers.

In some embodiments, only a single kind of particles 22 is used. In some embodiments, two or more kinds of particles 22 are used. The kinds of the particles 22 can be optional. For example, the particles 22 can be two kinds of the metal particles, or one kind of the metal particles and one kind of the metal oxide particles, or one kind of the metal oxide particles and one kind of the silicon oxide particles, or two kinds of the silicon oxide particles. These are for the purpose of example only and not intended to limit the present disclosure.

The aforementioned solvent 24 is, for example, but not limited to, a polar solvent (e.g., water or another polar solvent, such as tetrahydrofuran (THF), dimethyl sulfoxide (DMSO), dimethyl formamide (DMF) or acetone), an alcoholic solvent (e.g., methanol or ethanol), an aromatic solvent (e.g., toluene, benzene, xylene, or another aromatic solvent), a non-polar solvent (e.g., methyl ethyl ketone (MEK)), chloroform, or a combination thereof.

In some embodiments, an auxiliary material may be added to the mixed liquid 20, and the auxiliary material is used to adjust a distance between the particles of a particle layer in Step S104. The auxiliary material can be, for example, plastic particles or resin, which can be dissolved by a subsequent medium material or encapsulated into the medium material.

After the mixed liquid 20 is formed (Step S102), the mixed liquid 20 is placed on a substrate 12 to form a particle layer 202 (Step S104). In some embodiments, as shown in FIG. 2, the mixed liquid 20 is poured into a culture vessel 10 including the substrate 12, but another method may also be used to place the mixed liquid 20 into the culture vessel 10, such as coating, spraying or another suitable method. In some embodiments, the substrate 12 is a glass sheet or a plastic sheet, but is not limited thereto. In one embodiment, the culture vessel 10 may be, for example, but not limited to, a petri dish, a multi-well plate having at least 6 wells (6-well), or a multi-well plate having up to 384 wells (384-well).

In some embodiments, before the mixed liquid 20 is placed on the substrate 12 (Step S104), hydrophilization pretreatment is performed on the substrate 12, and the hydrophilization pretreatment includes surface plasma treatment, hydrophilic polymer coating, acid or alkaline rinse or a combination thereof. The surface plasma treatment is, for example, oxygen plasma or atmospheric plasma. The hydrophilic polymer coating is, for example, polyester polymer coating, such as poly(2-hydroxyethyl methacrylate), zwitterionic polymer or polyethylene glycol. The acid or alkaline rinse may be using hydrochloric acid, acetic acid or sodium hydroxide aqueous solution.

In some embodiments, after the mixed liquid 20 is placed on the substrate 12 (Step S104), standing treatment is performed to make the one or more kinds of particles 22 of the mixed liquid 20 self-assemble and arrange to form the particle layer 202. Time of the standing treatment is not limited, as long as the particles 22 in the liquid layer 201 can be self-assembled and arranged, or the solvent 24 can be further partially or completely volatilized. The term “self-assemble and arrange” mentioned here means that the particles 22 in the liquid layer 201 are automatically and substantially regularly arranged on the substrate 12, and distances between the particles 22 are maintained in a certain range, and the distances are based on the selected size(s) of the particles 22, and the distances between the particles are in a range of between zero and three times the particle diameter. For example, the diameter of the particles 22 is 10 micrometers, and the distance between the particles after self-assembly and arrangement can be in a range of between 0 micrometers and 30 micrometers.

In some embodiments, drying treatment is performed after the standing treatment is performed, and the drying treatment includes dehumidification drying, reduced pressure drying, heating drying or a combination thereof. The drying process is used to completely evaporate the solvent 24, leaving the particles 22 (i.e., the particle layer 202 is formed).

After the particle layer 202 is formed (Step S104), a medium material 26 is added to the particle layer 202 (Step S106). In some embodiments, as shown in FIG. 2, the medium material 26 is poured into the culture vessel 10, but another method may also be used to place the medium material 26 into the culture vessel 10, such as coating, spraying, or another suitable method. In some embodiments, as shown in FIG. 2, the medium material 26 does not completely cover the particle layer 202, and portions of surfaces of the particles 22 are exposed.

The medium material 26 is selected from the group consisting of styrene and its derivatives, polyester monomers, silicon oxide compounds, and combinations thereof. The styrene derivatives include carboxylated styrene, styrene sulfonic acid, or a combination thereof. The polyester monomers include methylmethacrylate. The silicon oxide compounds include organosilicon oxide compounds, such as polydimethylsiloxane, tetraethoxysilane, or a combination thereof.

After the medium material 26 is added to the particle layer 202 (Step S106), the medium material 26 is polymerized to form a medium layer 26′ to fix the particle layer 202 on the substrate 12 (Step S108), so that a composite material film 203 including the particle layer 202 and the medium layer 26′ is formed. The polymerization method includes, but is not limited to, free-radical polymerization, cationic polymerization, anionic polymerization, or condensation polymerization. In some embodiments, the medium material 26 can be polymerized and cured to form the medium layer 26′ by initiating a polymerization reaction by heating or ultraviolet light. The medium layer 26′ includes polystyrene and its derivatives (e.g., polycarboxylated styrene or polystyrene sulfonic acid), polyester (e.g., polymethyl methacrylate), silicon dioxide, silica gel, silicone, silicone rubber or a combination thereof. The composite material film 203 is experimentally confirmed to have performance of expanding circulating tumor cells with high efficiency and to have high reliability and high stability in operation.

The present disclosure further provides a composite material film for expanding circulating tumor cells ex vivo. As shown in FIG. 2, the composite material film 203 includes the particle layer 202 and the medium layer 26′ between the particles 22 of the particle layer 202 (i.e., filled in gaps between the particles 22).

The particle layer 202 includes one or more kinds of particles 22 selected from the group consisting of metal particles, metal oxide particles, silicon oxide particles, and combinations thereof.

The medium layer 26′ is selected from polystyrene and its derivatives (e.g., polycarboxylated styrene or polystyrene sulfonic acid), polyester (e.g., polymethyl methacrylate), silicon dioxide, silica gel, silicone, silicone rubber and combinations thereof.

Notably, as shown in FIG. 2, portions of surfaces of the one or more kinds of particles 22 are exposed and not covered by the medium layer 26′, which will facilitate attachment of the circulating tumor cells to the particles 22 and subsequent expansion.

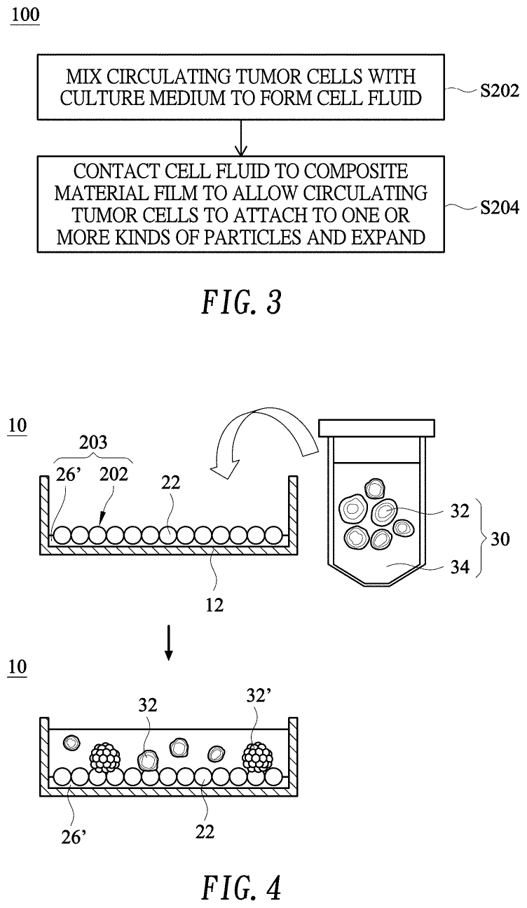

The present disclosure further provides a method for expanding circulating tumor cells ex vivo. FIG. 3 is a flow chart of a method 200 for expanding circulating tumor cells ex vivo according to some embodiments of the present disclosure. As shown in FIG. 3, the method 200 for expanding circulating tumor cells ex vivo includes following steps: mixing circulating tumor cells with a culture medium to form a cell fluid (Step S202) and contacting the cell fluid to the aforementioned composite material film to allow the circulating tumor cells to attach to the one or more kinds of particles and expand (Step S204). FIG. 4 is a schematic diagram of steps of a method for expanding circulating tumor cells ex vivo according to some embodiments of the present disclosure. For the following embodiments, please refer to FIG. 3 and FIG. 4 simultaneously.

First, circulating tumor cells 32 and a culture medium 34 are mixed to form a cell fluid 30 (Step S202). In some embodiments, the circulating tumor cells 32 are isolated from blood of an organism. In one embodiment, a separation procedure is performed on the blood of the organism to obtain peripheral blood mononuclear cells (PBMC) containing the circulating tumor cells 32, and a leukapheresis reagent in a form of an antibody is used to remove excess white blood cells in the peripheral blood mononuclear cells, and purified to cell size to obtain the circulating tumor cells 32. The blood source of the above-mentioned organism can be human or other animals, such as cats, dogs or other domesticated mammals. The circulating tumor cells 32 are, for example, but not limited to, tumor cells from small cell lung, lung, breast, pancreatic, sarcoma, melanoma, liver, esophagus, colorectal, nasopharyngeal, or brain cancers.

In one embodiment, the culture medium 34 includes a stem cell culture medium. As for other components in the culture medium 34, appropriate components can be used according to the type of the circulating tumor cells 32. In some embodiments, the culture medium 34 includes a basal culture medium, such as MEM, DMEM, RPMI1640, or another suitable basal medium. In some embodiments, the culture medium 34 also contains antibiotics to prevent microbial and fungal contamination. In one embodiment, the culture medium 34 further includes one or more recombinant growth factors, such as a basic fibroblast growth factor, an epidermal growth factor, and other supplements mentioned in the published literature to support growth of the circulating tumor cells. In some embodiments, the culture medium 34 includes a platelet lysate.

After the cell fluid 30 is formed (Step S202), the cell fluid 30 is brought into contact with the composite material film 203, so that the circulating tumor cells 32 are attached to the particles 22 and expanded (Step S204). As shown in FIG. 4, circulating tumor cell aggregates 32′ can be formed after expansion of the circulating tumor cells 32.

The expanded circulating tumor cells 32 and circulating tumor cell aggregates 32′ can be used to evaluate personalized drug candidates. Accordingly, the present disclosure provides a method for detecting an effect of a drug, including: adding a drug to the expanded circulating tumor cells 32 and the circulating tumor cell aggregates 32′, and then examining viability of the circulating tumor cells 32 and the circulating tumor cell aggregates 32′. From this, it can be determined whether the drug can reduce the viability of the circulating tumor cells 32. After a variety of drugs (which can be known drugs or new drugs) are detected by the above method, one of the drugs that can most significantly reduce the viability of the circulating tumor cells 32 can be selected as the preferred drug for the treatment of cancer, or personalized advice on medication selection can be provided.

The present disclosure further provides a kit for expanding circulating tumor cells ex vivo, which includes a culture vessel and a culture medium. Referring to FIG. 4, the kit includes a culture vessel 10 and a culture medium 34. The culture vessel 10 includes a substrate 12 and a composite material film 203 (including a particle layer 202 and a medium layer 26′) attached to the substrate 12. The culture medium 34 includes a stem cell culture medium. For the embodiments of the culture medium 34, please refer to the above, which will not be repeated here. After this kit is obtained, it can be used with circulating tumor cells to efficiently and stably expand the circulating tumor cells ex vivo.

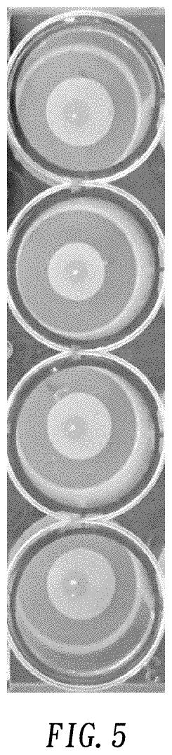

FIG. 5 is an image of a composite material film of Experimental Example 1 of the present disclosure. The preparation method of the composite material film of Experimental Example 1 included: forming a mixed liquid containing particles; performing hydrophilization pretreatment on a substrate; placing the mixed liquid containing the particles on the pre-hydrophilized substrate to form a particle layer; adding a medium material to the particle layer; and subjecting the medium material to a polymerization reaction. As shown in FIG. 5, the particle layer was not damaged and had a uniform thickness and had good adhesion to the substrate. From this, it can be seen that the substrate pretreated with the hydrophilization contributed to the formation of the particle layer with good quality and good adhesion to the substrate.

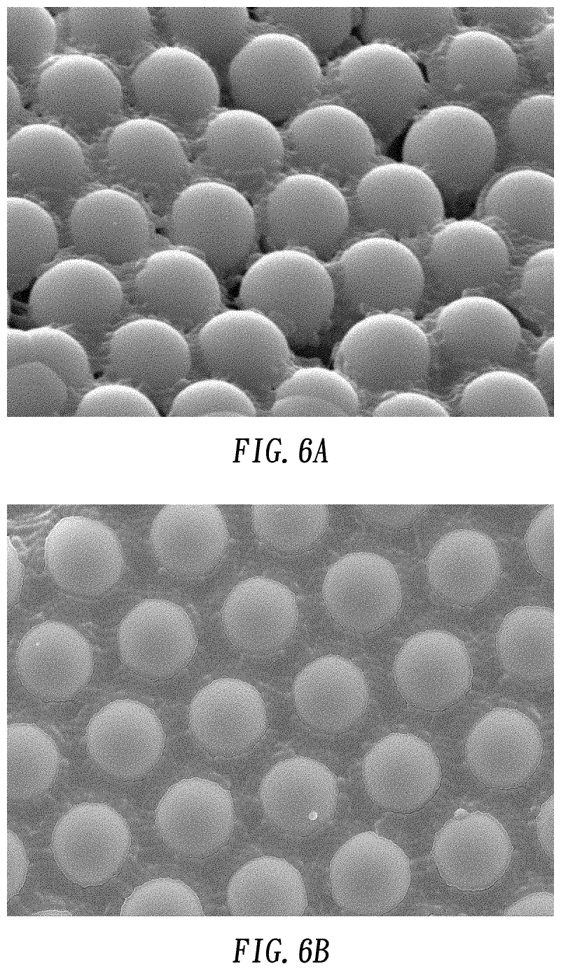

FIG. 6A is a SEM image of a material film of Comparative Example 1 of the present disclosure. The difference between Comparative Example 1 and Experimental Example 1 is that the preparation method of Comparative Example 1 did not include steps of adding a medium material to the particle layer and subjecting the medium material to a polymerization reaction. In other words, the material film of Comparative Example 1 had no medium layer. As shown in FIG. 6A, there were holes between some of the particles, which would cause the particles to fall off easily, and it was not conducive to the attachment and expansion of the circulating tumor cells and subsequent analysis.

FIG. 6B is a SEM image of a composite material film of Experimental Example 1 of the present disclosure. As shown in FIG. 6B, the medium layer between the particles was complete without any voids.

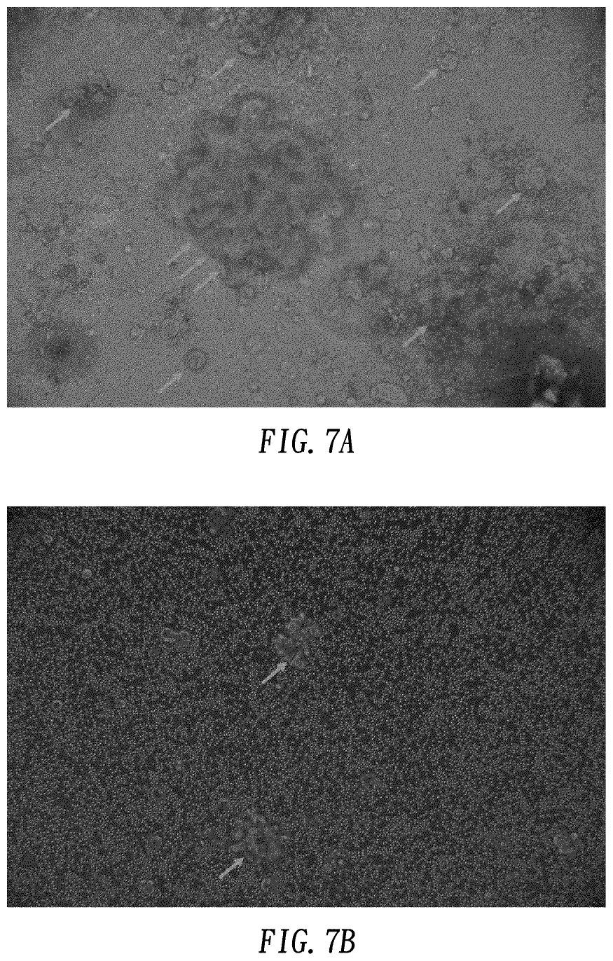

FIG. 7A is an optical microscope image of cell morphology of breast cancer cells after being cultured on the material film of Comparative Example 1. FIG. As shown in FIG. 7A, there were colonies formed by proliferation of the circulating tumor cells and the circulating tumor cell aggregates on the material film of Comparative Example 1 (indicated by arrows in the figure).

FIG. 7B is an optical microscope image of a cell fluid collected in a clean culture plate after the breast cancer cells cultured on the material film of Comparative Example 1 is rinsed. Taking a 24-well plate as an example, rinsing conditions were 20 mL of phosphate-buffered saline and a flow rate of 1 mL/sec. As shown in FIG. 7B, the cells were successfully collected after rinsed, but a large number of the particles fell off, which would affect subsequent detection and analysis.

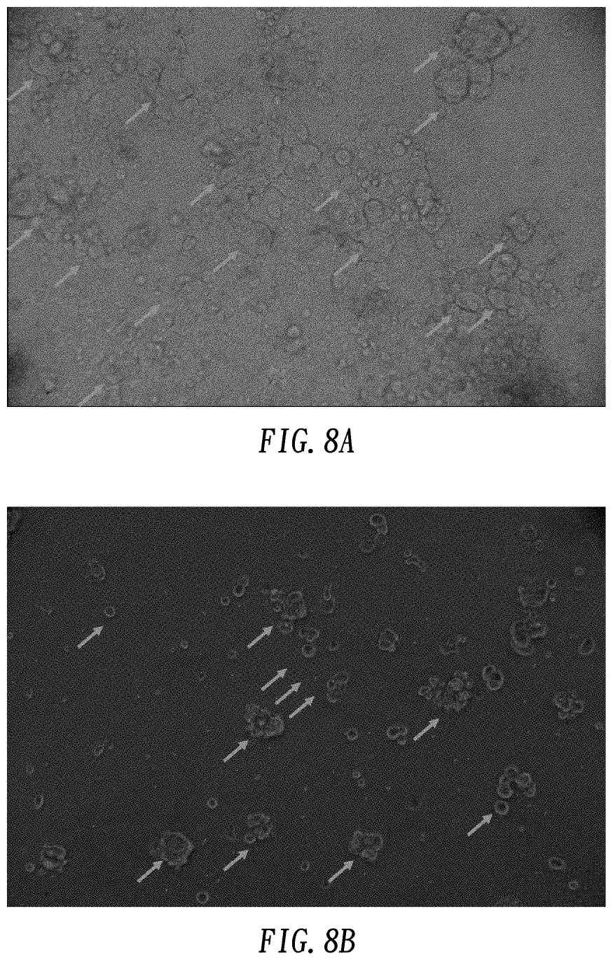

FIG. 8A is an optical microscope image of cell morphology of breast cancer cells after being cultured on the composite material film of Experimental Example 1. As shown in FIG. 8A, there were colonies formed by proliferation of the circulating tumor cells and the circulating tumor cell aggregates on the composite material film of Experimental Example 1 (indicated by arrows in the figure).

FIG. 8B is an optical microscope image of a cell fluid collected in a clean culture plate after the breast cancer cells cultured on the composite material film of Experimental Example 1 is rinsed. The rinsing conditions were the same as above. As shown in FIG. 8B, the cells were successfully collected after rinsed, and only very few particles fell off. It can be seen that the particle layer of the composite material film of Experimental Example 1 not only provided the attachment and expansion of the circulating tumor cells, but also maintained a good state in the subsequent process (e.g., repeated rinsing) and had excellent reliability.

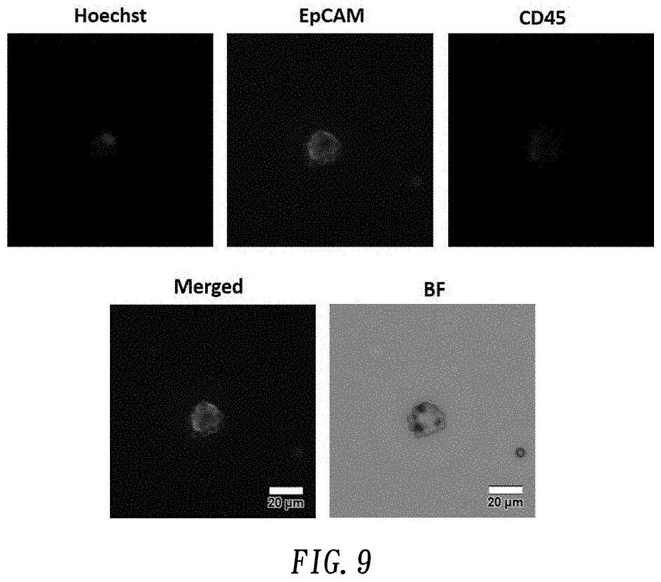

FIG. 9 shows characterization and identification of staining results of circulating tumor cells of a lung cancer patient after has been cultured on the composite material film of Experimental Example 1 of the present disclosure for the fourth week. In the immunofluorescence staining photos, EpCAM showed green fluorescence, and CD45 showed red fluorescence, and DPAI showed blue fluorescence. It can be found that the expanded cells still retained common EpCAM characteristics and no CD45 signal, so possibility that the cells were PBMC-related cells could be ruled out.

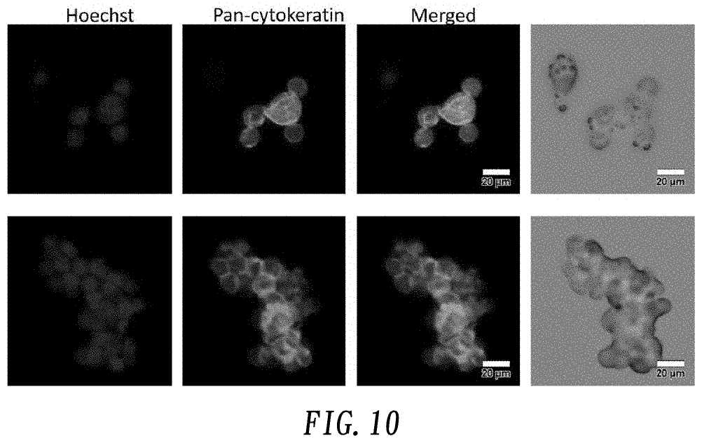

FIG. 10 shows another characterization and identification of staining results of the circulating tumor cells of the lung cancer patient after has been cultured on the composite material film of Experimental Example 1 of the present disclosure for the fourth week. In the immunofluorescence staining photos, Pan-cytokeratin showed green fluorescence, and DPAI showed blue fluorescence. Again, the expanded cells still had tumor cell characteristics.

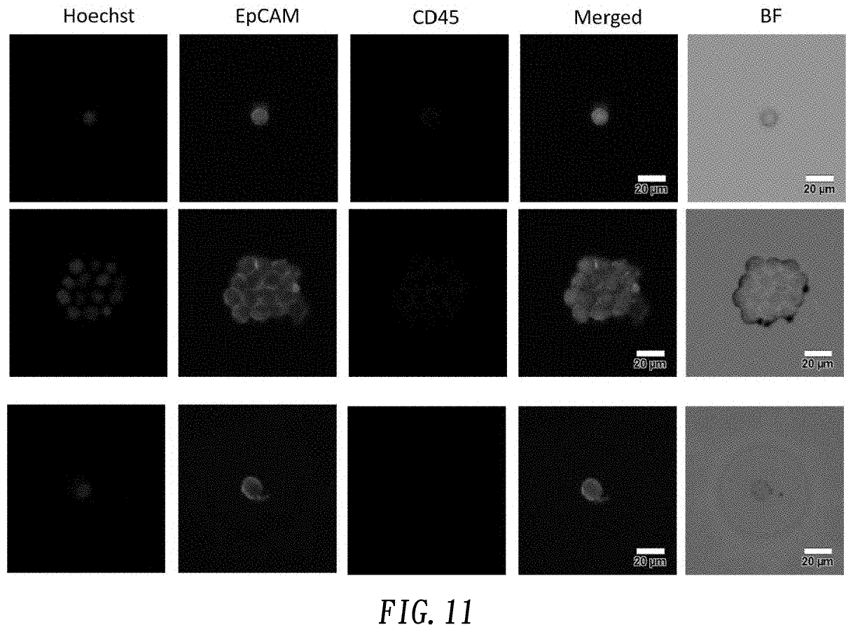

FIG. 11 shows characterization and identification of staining results of circulating tumor cells of a gastric cancer patient after has been cultured on the composite material film of Experimental Example 1 of the present disclosure for the fourth week. In the immunofluorescence staining photos, EpCAM showed green fluorescence, and CD45 showed red fluorescence, and DPAI showed blue fluorescence. It can be seen that the expanded circulating tumor cells still showed fluorescent signals of epithelial cell adhesion molecule (EpCAM) and DAPI, which proved that the expanded cells had tumor cell characteristics, but did not show fluorescence signals of common characteristics of T cells and B cells (CD45).

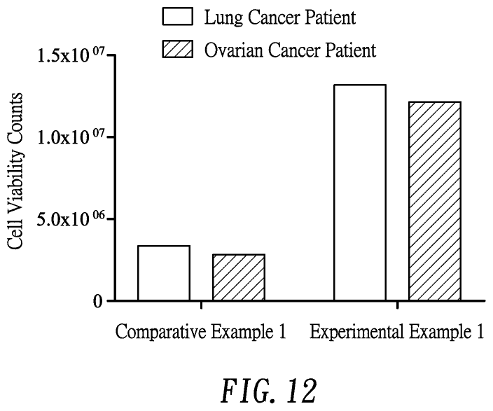

FIG. 12 is a graph showing comparison of cell viability counts after circulating tumor cells of a lung cancer patient and circulating tumor cells of an ovarian cancer patient have been cultured on the material film of Comparative Example 1 and the composite material film of Experimental Example 1 for four weeks. As shown in FIG. 12, the composite material film of Experimental Example 1 was able to effectively increase the number of the circulating tumor cells, so it could be seen that the composite material film of the present disclosure was quite suitable as a base for the circulating tumor cells to attach and expand.

The present disclosure further provides a cryopreservation solution for cryopreserving expanded circulating tumor cells, which includes a frozen reagent and a culture medium, and the culture medium includes a basic fibroblast growth factor (bFGF) and an epidermal growth factor (EGF). In some embodiments, the culture medium further includes a platelet lysate.

The cryopreservation solution can be mixed with the expanded circulating tumor cells and cryopreserved at a temperature below or equal to −70° C. (e.g., liquid nitrogen). Experiments have found that the thawed circulating tumor cells can restore their growth activity on the surface of the composite material film of the present disclosure, and their genetic material and biochemical properties have not been changed before and after being frozen. Therefore, the circulating tumor cells can still be applied in the above-mentioned detection method of drug effect even after being frozen, and can be further applied to a test of a drug poisoning effect in a process of new drug development.

In some embodiments, the culture medium includes 10 ng/ml of the basic fibroblast growth factor, 10 ng/ml of the epidermal growth factor, and 3%-20% platelet lysate. In some embodiments, a basal fluid of the culture medium is DMEM/F12 medium, and 10 ng/ml of the basic fibroblast growth factor, 10 ng/ml of the epidermal growth factor and 10% platelet lysate are added to the DMEM/F12 medium.

In some embodiments, the culture medium also includes one or more recombinant growth factors, such as supplements mentioned in other published literature to support growth of the circulating tumor cells.

In some embodiments, the culture medium further includes additives, such as B27 supplement. In some embodiments, the culture medium further includes MEM, RPMI1640, other suitable basal medium, or a combination thereof. In some embodiments, the culture medium further includes antibiotics to prevent microbial and fungal contamination.

Finally, it should be noted that the above embodiments are only used to illustrate the technical solutions of the present disclosure, but not limit them; although the present disclosure has been described in detail with reference to the foregoing embodiments, the person skilled in the art should understand that it is still possible to modify the technical solutions described in the foregoing embodiments, or to perform equivalent replacements for some technical features; and these modifications or replacements do not make the essence of the corresponding technical solutions deviate from the spirit and scope of the technical solutions of the embodiments of the present disclosure.

What is claimed is:

1. A method for preparing a composite material film for expanding circulating tumor cells ex vivo, comprising:
mixing one or more kinds of particles and a solvent to form a mixed liquid, wherein the one or more kinds of particles are selected from the group consisting of metal particles, metal oxide particles, silicon oxide particles and combinations thereof;
placing the mixed liquid on a substrate to form a particle layer;
adding a medium material to the particle layer, wherein the medium material is selected from the group consisting of styrene and its derivatives, polyester monomers, silicon oxide compounds and combinations thereof; and
polymerizing the medium material to form a medium layer to fix the particle layer on the substrate.

2. The method of claim 1, wherein the metal particles are selected from the group consisting of gold particles, silver particles, titanium particles and combinations thereof, and the metal oxide particles are titanium dioxide particles, and the silicon oxide particles are selected from the group consisting of silicon dioxide particles, silica gel particles, polydimethylsiloxane particles and combinations thereof.

3. The method of claim 1, wherein a particle size of the one or more kinds of particles is in a range of between 10 nanometers and 10 micrometers.

4. The method of claim 1, further comprising:
performing hydrophilization pretreatment on the substrate before placing the mixed liquid on the substrate, wherein the hydrophilization pretreatment comprises surface plasma treatment, hydrophilic polymer coating, acid or alkaline rinse or a combination thereof.

5. The method of claim 1, further comprising:
performing standing treatment after placing the mixed liquid on the substrate to make the one or more kinds of particles of the mixed liquid self-assemble and arrange to form the particle layer.

6. The method of claim 5, further comprising:
performing drying treatment after performing the standing treatment, and the drying treatment comprises dehumidification drying, reduced pressure drying, heating drying or a combination thereof.

7. The method of claim 1, wherein the styrene derivatives comprise carboxylated styrene, styrene sulfonic acid or a combination thereof, and the polyester monomers comprise methylmethacrylate.

8. The method of claim 1, wherein the silicon oxide compounds are selected from the group consisting of polydimethylsiloxane, tetraethoxysilane and combinations thereof.

9. A composite material film for expanding circulating tumor cells ex vivo, comprising:
a particle layer comprising one or more kinds of particles substantially regularly arranged, wherein the one or more kinds of particles are selected from the group consisting of metal particles, metal oxide particles, silicon oxide particles and combinations thereof; and
a medium layer disposed between the one or more kinds of particles of the particle layer, wherein the medium layer is selected from the group consisting of polystyrene and its derivatives, polyester, silicon dioxide, silica gel, silicone, silicone rubber and combinations thereof,
wherein surfaces of some of the one or more kinds of particles are partially exposed and not covered by the medium layer.

10. A kit for expanding circulating tumor cells ex vivo, comprising:
a culture vessel, comprising:
a substrate; and
the composite material film prepared by the method of claim 1, attached to the substrate; and
a culture medium, comprising a stem cell culture medium.

11. A method for expanding circulating tumor cells ex vivo, comprising:
mixing a plurality of circulating tumor cells with a culture medium to form a cell fluid; and
contacting the cell fluid to the composite material film prepared by the method of claim 1 to allow the circulating tumor cells to attach the one or more kinds of particles and expand.

12. A method for detecting an effect of a drug, comprising:
adding a drug to the expanded circulating tumor cells of the method of claim 11; and
examining viability of the circulating tumor cells.

13. A cryopreservation solution for cryopreserving expanded circulating tumor cells, comprising:
a frozen reagent; and
a culture medium, comprising a basic fibroblast growth factor (bFGF) and an epidermal growth factor (EGF).
